(19) United States
(12) Patent Application Publication
(10) Pub. No.: US 2010/0004564 A1
(43) Pub. Date: Jan. 7, 2010

(54) WOUND MEASURING DEVICE

(76) Inventor: Johan Jendle, Hammaro (SE)

Correspondence Address: Manelii Denison & Selter PLLC, 2000 M Street, 7th Floor, Washington DC, DC, 20036, US

(21) Appl. No.: 12/374,797

(22) PCT Filed: Jul. 25, 2006
(86) PCT No.: PCT/SE06/50273

§ 371 Date: Jan. 23, 2009

(51) Int. Cl. A61B 5/107

(52) U.S. Cl. 600/587

(57) ABSTRACT

This invention relates to a wound measuring device, comprising a transparent, flexible and bendable sheet (1) with at least a first edge (3), a second edge (4) and a third edge (5) and a cross-ruled field (7) located on the transparent sheet (1), wherein at least adjacent one edge (5) there is a ruler (12A) and the cross-ruled field (7) comprises a number of equally sized identically shaped fields (13, 14), wherein at least one corner (6) formed by two of said edges (3, 5) form an angle (α) which is less than 90°.

### FIELD OF INVENTION

This invention relates to a wound measuring device, comprising a transparent, flexible and bendable sheet with at least a first edge, a second edge and a third edge and a cross-ruled field located on the transparent sheet, wherein at least adjacent one edge there is a ruler and the cross-ruled field comprises a number of equally sized identically shaped fields.

### BACKGROUND OF THE INVENTION

Superficial and primary banal wounds of the skin can have serious consequences, especially in some subjects prone to infections i.e. patients with diabetes mellitus or with impaired immunity.

A majority of wounds; small abrasive wounds, pressure ulcers, minimal lacerations, small cuts or small thorns could all initiate a deep infection and even develop into a gangrenous process. Pressure ulcers develop when a tissue is exposed to a pressure that leads to tissue damage. Pressure ulcers in patients with various neuromuscular diseases are at present a large and expensive problem in the field of medicine. Diabetic foot ulcers occur in up to 15% of all diabetic patients and are a leading cause of non-traumatic amputation in these patients.

The earlier a wound is discovered and treated the less is the likelihood of deep tissue damage and the shorter is the time to healing.

A number of different strategies are involved in promotion of wound healing. Independent of the treatment used the follow-up of the wound healing must be assessed on a regular basis and the size of the wound as well as the depth should be measured.

The wound must be measured repeatedly, often with short intervals, to evaluate the current therapy. The size of the wound as well as the depth is important as prognostic markers and to decide whether the patient needs to be referred to a specialist. Different persons often preform the follow-up examination. This has a negative effect on the precision of the measurements.

Various methods are used clinically to measure wounds. Usually a common ruler is being used. The ruler is held just above the skin surface and by moving the ruler both the length and width of the wound can be estimated. A slide calliper could also be used for this purpose. Both these methods are for multiple uses.

Bacteria often colonize wounds. These bacteria usually originate from the skin or gut of the patient and have spread to the wound. There is also a risk that the medical staff might contaminate and cause a secondary infection of a small wound when assessing the healing process. The risk of contamination further increases when a multiple ruler is being used to measure the wound. There is a risk of contaminating a different wound on the same patient and/or transferring bacteria from patient to patient.

There are also known different kind of single use measuring devices. From EP 0730845 and U.S. Pat. No. 5,265,605 there are known devices comprising a transparent sheet that can be applied on the surface of the wound and the contour of the wound with indicia that can be used to estimate the area of the wound. These known sheets, however, are in the form of double layers, being separable, which implies complications in use and also increased cost. Moreover they do not allow measurement of depth of wound. Accordingly supplementary probes are used for measuring the depth of a wound, e.g. probes that are for single use.

Digital photos of wounds is another known method that allows assessment of the size of the wound. However, a comparison could be difficult depending on the distance, type of camera etc, and it cannot be used to measure the depth. Techniques to measure the volume of wounds have been described using 3-dimensional pictures, however these techniques have not been used in clinical practice, probably because of the inherent complexity. Casting of the wounds and evaluation of the volume of wounds by injecting fluids and gels into wounds have been described, these methods are time-consuming and not used in clinical practice.

### OBJECT OF THE INVENTION

It is an object of the invention to eliminate or at least minimize the above problem/s, which is achieved by means of a wound measuring device, comprising a transparent, flexible and bendable sheet with at least a first edge, a second edge and a third edge and a cross-ruled field located on the transparent sheet, wherein at least adjacent one edge there is a ruler and the cross-ruled field comprises a number of equally sized identically shaped fields, wherein at least one corner formed by two of said edges form an angle which is less than 90°.

Thanks to the invention there is provided a cost-effective single purpose measuring device that is easy to use with high accuracy and allows a safe and accurate measurement of the size (diameter, area and depth) of a skin wound without increasing the risk of secondary infection.

By the use of a single disposal measuring device for the measurement of wounds the risk of contamination can be reduced.

### BRIEF DESCRIPTION OF THE DRAWINGS

In following the invention will be described in more detail with reference to the annexed drawings, wherein:

FIG. 1 shows a preferred embodiment of wound measurement device according to the invention,

FIG. 2 shows a first modification of the device according to the invention,

FIG. 3 shows a second modification according to the invention,

FIG. 4 shows a third modification according to the invention,

FIGS. 5-7 show the same basic design as showing in FIG. 1, but the use of different patterns within the cross field, and

FIGS. 8-13 show further patterns that may be used to measure surface area in accordance with the invention.

### DETAILED DESCRIPTION

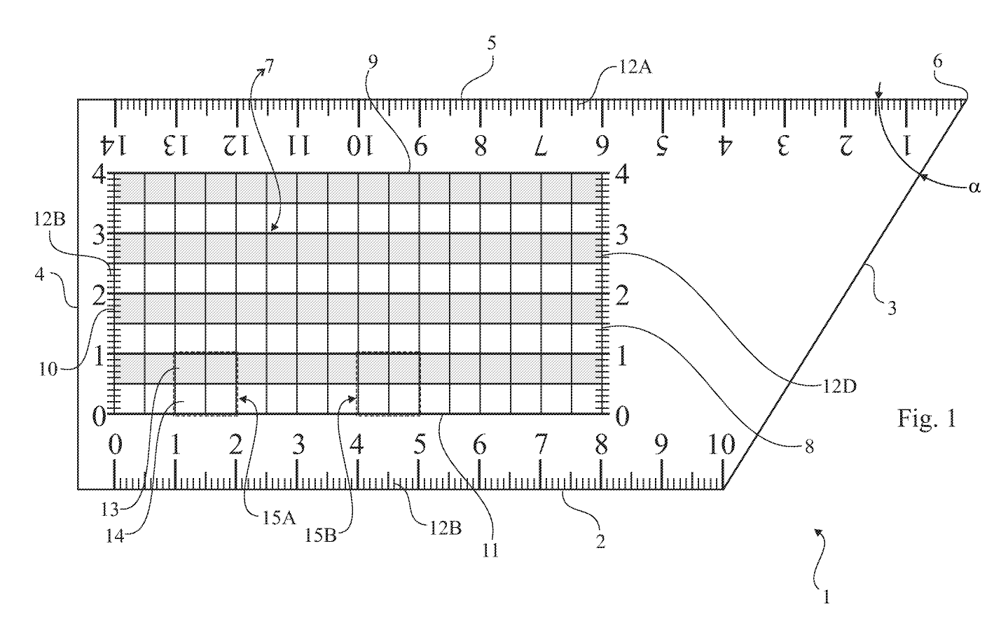

In FIG. 1 there is shown a preferred embodiment of measuring device according to the invention. The device is made from a polymeric material that is bendable, flexible, and with resilient properties. The sheet material 1 is very thin, preferably about 0.25 mm (0.1-2 mm), and translucent, to allow for a wound to be clearly visually apparent when looking through the device 1. The sheet 1 has three rectilinear edges 2, 4, 5 and one sloping edge 3. Along both of the side edges 2, 5 (which are parallel) there are positioned rulers 12A, 12B. One of these edges 5 is longer than the other 2. This is achieved by arranging an upper edge 3 at an angle α, in relation to the line of direction of the side edges 2, 5, to form a sharp angle α at the corner 6 between the longest side edge 5 and the upper edge 3 of about 60°. It is to be noted that the ruler 12A that is positioned at the longest side edge 5 is positioned co-linear with the side edge 5. Hereby the start of the ruler 12A may be chosen to conform exactly with the sharp corner 6.

This longest side edge 5 is at least 10 cm long, preferably longer than 10 cm and normally about 14-15 cm. The shorter side edge 2, which presents a second ruler 12B is preferably about 8-12 cm, more preferred about 10 cm. A lower edge 4 is positioned perpendicularly in relation to the side edges 2, 5.

Onto the inner surface of the flexible sheet 1, there is positioned a cross field 7. The cross field has a rectangular shape presenting two side borders 9, 11 which run parallelly with the side edges 2, 5 and a bottom border 10 and a top border 8. In the shown embodiment the side borders 9, 11 are longer than the other borders 8, 10, e.g. presenting a field that is 4 times 8 cm. At each one of the lower border 10 and the top border 8 there are positioned a third 12C and a fourth 12D ruler respectively. Further it is shown that the cross field 7 is formed by means of a number of squares 13, 14, which according to the preferred embodiments have a size of 5*5 mm (0.5*0.5 cm=0.25 cm²). Further it is shown that some of the fields 14 has a first visual appearance (e.g. totally translucent) and a second kind of field 13 has a second kind of visual appearance (e.g. different colour, for example greyish). Further it is shown that the fields 13, 14 do form groups 15A, 15B. Each group 15A, 15B does contain a number of fields 13, which form a pattern within one group 15A that is identical with any other group 15B. In this embodiment that is achieved by having every second horizontal area of fields 13 presenting a different colour than the horizontal fields 14 them between. As a consequence within each group 15A, 15B there will be two grey coloured fields 13 and two totally transparent (uncoloured) fields.

In use the sharp corner 6 will be used to measure the depth of a wound. Thanks to having a sharp angle α it will be possible to allow the corner 6 to penetrate into the wound to thereby facilitate to read the depth/thickness of the wound. Further the sheet 1 is used to measure the surface area of the wound, which is achieved by positioning the cross field 7 on top of the wound. Thanks to the use of differently coloured fields 13, 14 it is easily achieved to make a quick estimate of the surface area, since the user will know that each group 15A, 15B equals 1 cm² and that each field 13, 14 equals 0.25 cm². Hence the user may easily first estimate the larger rectangular area within the cross field 7 that the wound covers and there after easily add the surface area represented by the unregular protruding portions of the wound.

Thanks to the use of very thin flexible sheet 1 the material costs for the sheet is extremely low. Further the cost of production thereof may be very low since the sheet allows for very cost-efficient printing of the rulers 12A-12D and cross field 7 respectively, e.g. by means of screen print or digital etching technique. Hence, they can be disposed of without causing any significant impact on cost.

The material used could be chosen from many different polymers, as is well understood by a skilled person within the field of polymers, e.g. acrylic, polystyrene, A-polyethylene biodegradable, polyvinylchloride, polycarbonate. Indeed it may be preferable to use a polymer that is biodegradable, e.g. A-polyethylene. In a tested and appreciated embodiment PVC was used, due to being kind to the skin and having good price-quality balance, having a thickness within the range 0.2-0.3 mm. However, depending on the stiffness of chosen material the thickness may vary within a wide range, e.g. 0.1-2 mm, but normally within the range of 0.2-0.5 mm. In the tests screen print has been successfully used to print the rulers, etc; whereby within food industry and for toys accepted solvent based paints/dyes have been used.

Thanks to making the sheets 1 disposable, it will eliminate any risk for hazardous contamination. Hence, the measuring device 1 will be thrown away after use. Moreover, since a very thin flexible sheet 1 may be used to obtain sufficient function the user may carry a relatively large number of sheets without causing any extra burden or occupying any large space. In the preferred embodiment the sheets are contained within a separate sealable plastic bag (or similar) that easily fits into a pocket. Thanks to the arrangement of the lower edge 4 it may comfortably be positioned in a pocket. Accordingly any user will have easy access to the wound measuring device. Moreover, according to the preferred embodiment of the invention the device may be beneficial used to print supplemental information on to it or any other kind of information that is useful to have nearby for users of the device. Hence, the invention presents a kind of multipurpose ruler that allows quick and safe measurement of various wounds and allows accurate assessment of size, area and depth of the wound. Areas from 0.25 to 32 cm² can readily be estimated as well as wound sizes ranging from 1 mm up to 140 mm.

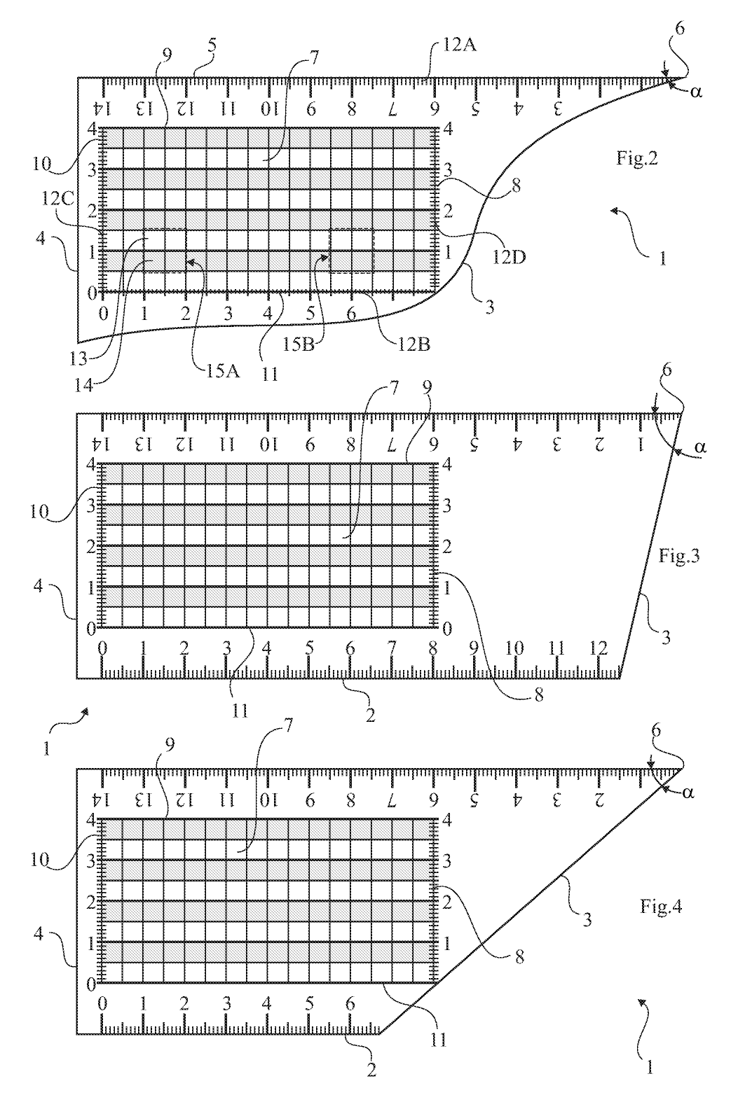

In FIGS. 2, 3 and 4 there are shown three alternative modifications within the scope of the invention. FIG. 2 shows that the sheet may have a triangular shape, i.e. merely presenting three side edges 3, 4, 5. Here it is also shown that the triangular shape may be slightly modified to allow for the cross field 7 to cover a larger area. Important, however, is that this modification shows that it is possible to achieved a very sharp angle α, e.g. being about 10°, as can be desired for some applications. A further modification shown in FIG. 2 is that the second ruler 12B has been positioned to be in line with the lower side border 11 of the cross field 7.

FIG. 3 shows that the invention may also be used having a much larger angle α than 60°, e.g. about 80°, which in some cases might be preferable. FIG. 3 shows that a very sharp angle α, (about 30°) may also be achieved in combination with using four side edges 2, 3, 4, 5.

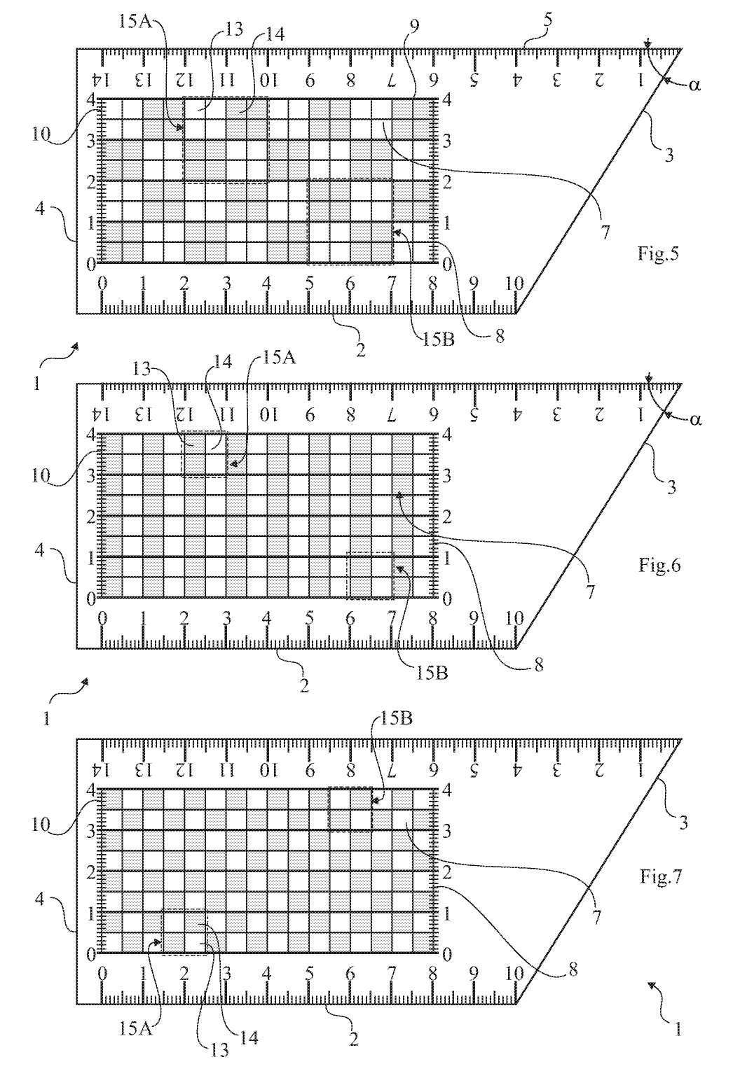

In FIG. 5 there is shown a sheet 1 which is substantially the same as the one shown in FIG. 1, except for a differently used pattern within cross-field 7. Here each group 15A, 15B is chosen to be much larger than the groups of FIG. 1, which is achieved by giving the same kind of visual appearance to four fields 13, 14 being positioned beside each other within a square. Accordingly there will be a first square of four totally transparent fields 13, which forms a total area of 1 cm², and next thereto there will be a second square of four coloured fields 14 also forming a square surface of 1 cm². Accordingly each group 15A, 15B will include four such squares, i.e. comprising in total 4 cm² and in total 16 fields.

In FIG. 6 there is shown a further embodiment, where the similarly coloured fields 13, 14 respectively are extending in a direction that is perpendicularly in relation to the side edges 2, 5. In other respects it is the same as shown in FIG. 1. Also FIG. 7 is similar as FIG. 1 except for a different grouping of the similarly coloured fields 13, 14. Here they are positioned diagonally in relation to the extension of the side edges 2, 5, such that a chesslike pattern is achieved.

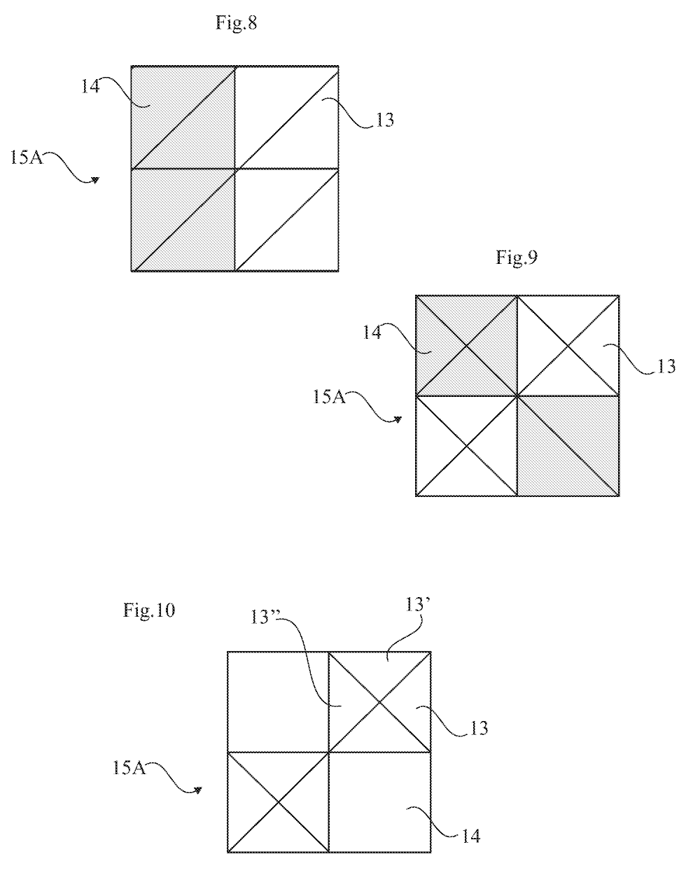

FIGS. 8-10 show different further patterns within the cross field 7. In these figures merely one group 15A is presented. In brief it is shown that also triangularly shaped fields 13, 14 may also be used, wherein the fields used in FIG. 9, 13, 14 are half the size the fields used in FIG. 8. It is evident that this manner of dividing the groups may efficiently be used to adopt the chosen surface area to different measuring systems, e.g. the use of FIG. 8 may suit the metric system, having the group 15A corresponding to 1 cm² whereas the group 15A shown in FIG. 9 may correspond to 1 in². In FIG. 10 it is shown that the fields 13, 14 may be given a different visual appearance by other means than colours, e.g. different kind of printed lines, to thereby possibly also form subfields 13′, 13″ within every second field 13.

In FIG. 11 it is shown that each group 15A must not form a square but, as in this case may form any kind of pattern that can be used to distinguish different fields 13, 14 and groups 15A from each other. In this case hexagonal fields 13, 14 are used and a group 15A consisting of three coloured fields 13 and four totally transparent fields 14. It is understood that the group could instead be chosen to have an equal number of coloured and transparent fields 13, 14 respectively, e.g. two coloured fields 13, 14 extending vertically next to each other and two transparent fields 14 also extending vertically next to each other (and next to the coloured fields 13).

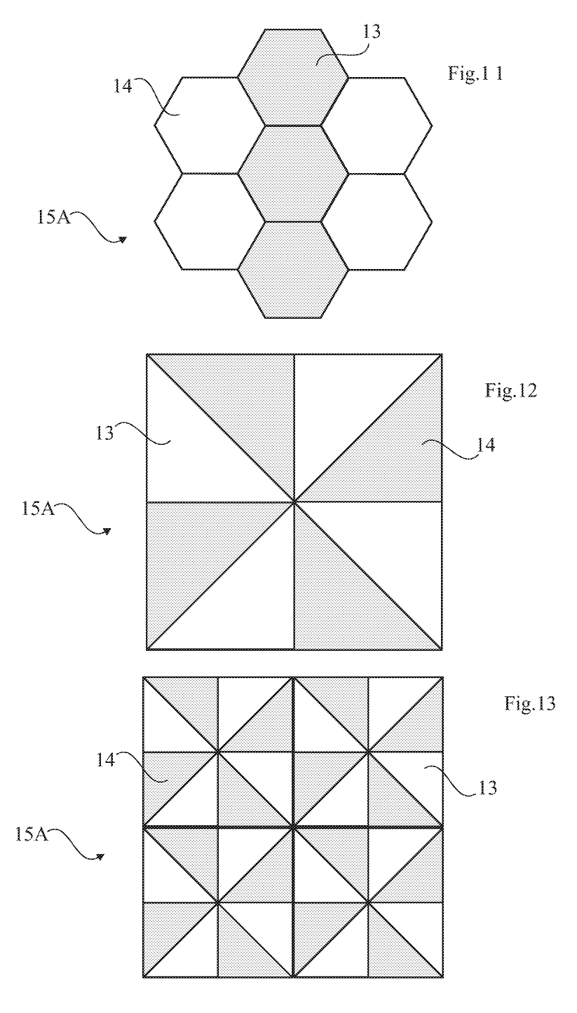

In FIGS. 12 and 13 again there is shown a different manner of forming a group 15A to present fields 13, 14 having triangular shape of different colours.

It is evident that the invention is not delimited by the embodiments that have been described above but may be varied within the scope of the appended claims. For instance, it is evident that many different kind of varying shapes along the lines indicated above may be used to achieve a beneficial function of measuring device according to the invention, not at least regarding the pattern of the cross field 7. For instance, not all fields have to be equally sized, but having every second field split in half to there by also provide the visual difference. Moreover it is evident that the positioning of rulers 12A-12D may also be varied within wide frames to adapt to different needs of different individuals and/or situations. Moreover the skilled person realizes that many kind of different materials may be used to produce the sheet 1 for the invention, fulfilling the needs thereof. Also many different kind of printing techniques may of course be used to achieve a device according to the invention.

1-13. (canceled)

14. Wound measuring device comprising:
a transparent, flexible and bendable sheet having at least a first edge, a second edge and a third edge, and a cross-ruled field located on the transparent sheet, wherein at least adjacent one edge there is a ruler and the cross-ruled field comprising a number of equally sized identically shaped fields, at least one corner formed by two of said edges forms an angle (α) which is less than 90°.

15. Wound measuring device according to claim 14, wherein said shaped fields are arranged to form at least some identical groups and that within each group there is at least one field that is visually distinguishable from a neighbouring field.

16. Wound measuring device according to claim 14, further comprising a fourth edge.

17. Wound measuring device according to claim 14, wherein at least one edge of said cross-ruled field is in the form of a second ruler.

18. Wound measuring device according to claim 14, wherein said cross-ruled field comprises a first cross-ruled edge, a second cross-ruled edge, a third cross-ruled edge and a forth cross-ruled edge.

19. Wound measuring device according to claim 14, wherein said angle (α) is greater than, or equal to 10°.

20. Wound measuring device according to claim 14, wherein said angle (α) is between 10° and 80°.

21. Wound measuring device according to claim 14, wherein said angle (α) is about 60°.

22. Wound measuring device according to claim 14, wherein the sheet has resilient, polymeric properties.

23. Wound measuring device according to claim 14, wherein said fields form at least one selected from the group consisting of rectangles, triangles, and hexagons.

24. Wound measuring device according to claim 15, wherein the groups form squares.

25. Wound measuring device according to claim 24, wherein said fields form a number of smaller squares within each group.

26. Wound measuring device according to claim 24, wherein said groups each contain at least four of said fields.

27. Wound measuring device according to claim 24, wherein at least one group equals to one cm² or one in².

28. Wound measuring device according to claim 14, wherein at least one of said edges is smaller than 80 mm.

29. Wound measuring device according to claim 14, wherein the cross-ruled field is in the form of a rectangle.

30. Wound measuring device according to claim 29, wherein a first field border is about 1.5-2.5 times the length of a perpendicularly extending second border.

31. Method for the measurement of wounds by using a wound measuring device comprising a transparent, flexible and bendable sheet having at least a first edge, a second edge and a third edge, and a cross-ruled field located on the transparent sheet, wherein at least adjacent one edge there is a ruler and the cross-ruled field comprising a number of equally sized identically shaped fields, at least one corner formed by two of said edges forms an angle (α) which is less than 90°, the method comprising:
placing the wound measuring device directly on the surface of a wound;
registering the length and width of the wound using the rulers;
registering the area of the wound by the use of the cross-ruled field;
placing the point of one ruler at the bottom of the wound; and
registering the depth by means of said ruler.
